(19) United States
(12) Patent Application Publication
(10) Pub. No.: US 2008/0053459 A1
(43) Pub. Date: Mar. 6, 2008

(54) Anti-Snoring Device

(76) Inventor: Neil Bradley Silker, Carlton, MN (US)

Correspondence Address: WILLIAM T. HELWIG, ATTORNEY AT LAW, RUDY LAW FIRM, 813 CLOQUET AVENUE, CLOQUET, MN, 55720, US

(21) Appl. No.: 11/470,319

(22) Filed: Sep. 6, 2006

(51) Int. Cl. A61F 5/56

(52) U.S. Cl. 128/848

(57) ABSTRACT

An inter-lip device for regulating mouth airflow to prevent snoring while sleeping. A canoe shaped horizontal pocket is formed by hinge-ably attaching two resilient panels together by three common edges, sized to fit between the lips without protruding into the mouth. An adhesive covering is applied to both outer faces of the pocket and a slit is cut in the middle of the front edge hinge. The pocket opens to the rear into the mouth. The lips are sealed to the adhesive about the pocket opening, holding the mouth closed while directing mouth breathing through the device and out the front slit opening. With the jaw consciously flexed, resting mouth airflow is comfortably allowed, however, a relaxed sleeper's jaw or a forced inhalation will collapse the slit opening, reducing or stopping airflow, causing the user to breath through their nose, thereby preventing the necessary mouth airflow needed to support snoring.

### BACKGROUND OF THE INVENTION

The invention relates to an anti-snoring device and specifically to an inter-lip device for regulating mouth airflow to reduce or prevent snoring while sleeping.

It has been estimated that 30-50% of adults suffer from snoring to some degree. Snoring is most commonly caused by air turbulence in the oral airway and the resultant vibration of the surrounding soft tissues. Snoring may also be a sign that a person is suffering from a more serious problem such as sleep apnea, being a common symptom of that disorder. However, the majority of people who snore do not suffer from sleep apnea, but suffer primarily from the stress snoring may cause in their relationships.

Millions of spouses, partners and children suffer through the night due to the annoying noise of the snorer. This can cause relationship problems because of the disrupted sleep of the sleeping partner and other household members, who may try to stop the person snoring or may simply lie awake wishing they were asleep. Snoring may thus impact the quality and quantity of sleep of other household members to an extent greater than it affects the snorer themselves. Snoring results in no one wanting to sleep with or near the snorer, and the snorer may become isolated. Such social ramifications, which can contribute to the breakdown in a relationship, make snoring a serious problem.

Snoring generally begins when a snorer's mouth falls open while sleeping and they begin to breath primarily through their mouth. Numerous patents have been developed to aid in the prevention of snoring, and in particular, to aid in the prevention of snoring by inhibiting mouth breathing during sleep, primarily by trying to prevent the mouth from falling open during sleep. The following patents have been found to be the most relevant in that endeavor:

U.S. Pat. No. 6,089,232 issued Jul. 18, 2000 to Portnoy et al. is a Snore Stopper, which is an elastic fabric panel with adhesive on one side, which adheres to and attaches over the mouth and lips of the user and prevents the mouth from opening while sleeping, but which includes a narrow slit cut through the middle of the panel to enable breathing and talking;

U.S. Pat. No. 7,055,526 issued Jun. 6, 2006 to Barakat et al. is an Anti-Snoring Device Comprising a Skin Compatible Adhesive, which is a flexible sheet of material that entirely covers the mouth and adheres to the skin surrounding the mouth to hold the mouth shut and promote nasal breathing;

U.S. Pat. No. 7,032,598 issued Apr. 25, 2006 to Portnoy is a Snoreway Space Block with Snore Strips or Portnoy Buccal Tab, which is a narrow adhesive strip that is applied from under the chin upwards across the mouth to underneath the nose, which holds the mouth shut to promote nasal breathing;

U.S. Pat. No. 5,873,363 issued Feb. 23, 1999 to Esmailzadeh is a Method and Apparatus for Breathing, which includes an oral barrier which is impervious to air and which has an adhesive backing, which is placed over the mouth to prevent airflow into the mouth and which may also be used in conjunction with a mouth closer;

U.S. Pat. No. 4,817,636 issued Apr. 4, 1989 to Woods is an Anti-Snoring Device, which is a sheet of flexible material with adhesive on the back face of the material, and which covers the users mouth completely, conforming around the mouth below the nose and cheeklines and above the chin, preventing the intake of air through the mouth;

U.S. Pat. No. 1,354,652 issued Oct. 5, 1920 to Jefferies is a Device to Prevent Mouth Breathing, which is a sheet of fabric material with adhesive on one face, which covers the users mouth completely, and attaches to the face above and below the lips and across to the cheeks to hold the mouth closed.

Other devices for the treatment of snoring are designed to be worn inside of the mouth, which are uncomfortable to wear, which are expensive, and which must be fitted and made to order. Such Intra-oral devices also often cause excessive salivation, dry mouth or temporomandibular joint (TMJ) discomfort. Other treatments for snoring include various surgeries, which are drastic steps to take to attempt to cure the problem, however snoring can be so disruptive to a person's life and relationships, that some sufferers resort to surgery.

All of the above listed patents refer to anti-snoring devices which prevent mouth breathing by blocking airflow through the mouth, or by holding the jaw or mouth shut, by making use of a material with adhesive on one side to attach over the mouth and partially across the face surrounding the mouth and thereby holding the lips together and the mouth shut. These type of devices are extremely uncomfortable and give the user the feeling that their face is taped up. The skin about the mouth and cheeks is pulled, tugged on and stretched with every movement, which can be a constant irritation to the user.

None of the above listed patents make use of a design which will automatically regulate airflow based on the users type of breathing and level of consciousness, to reduce or prevent snoring. None of the above listed patents make use of a design which is compact enough to fit between the lips. None of the above listed patents make use of a folded or hinged design which allows for some jaw movement for added comfort. None of the above listed patents make use of a design which attaches only to the lips to eliminate the pulling of skin across other portions of the face for added comfort, and without creating the feeling of having one's face taped up or one's mouth taped shut.

What is needed then is a device which will regulate airflow based on the users type of breathing and level of consciousness, constructed of a more rigid and resilient material, constructed using a folded design to fit between the lips for a compact design, creating a hinge in front with adhesive on the top and bottom faces to attach to the abutting lip surfaces where the device then attaches between the lips, not to the outside of the lips or the skin of the face surrounding the lips, for more comfortable use, holding the lips together and the mouth primarily closed without having the feeling of having one's face taped up or one's mouth taped shut.

### OBJECTS OF THE INVENTION

Accordingly, several objects and advantages of the invention are as follows:

It is an object of this invention to provide a simple device to prevent snoring.

It is an object of this invention to provide a compact device to prevent snoring.

It is another object of this invention to provide an inter-lip worn device to prevent snoring.

It is an object of this invention to provide a device which will regulate sleeping mouth airflow to prevent snoring.

It is an object of this invention to provide a comfortable device to prevent snoring.

Further objects and advantages will become apparent from a consideration of the following description and drawings.

### SUMMARY OF THE INVENTION

An inter-lip device for regulating mouth airflow to prevent snoring while sleeping. A canoe shaped horizontal pocket is formed by hinge-ably attaching two resilient panels together by three common edges, sized to fit between the lips without protruding into the mouth. An adhesive covering is applied to both outer faces of the pocket and a slit is cut in the middle of the front edge hinge. The pocket opens to the rear into the mouth. The lips are sealed to the adhesive about the pocket opening, holding the mouth closed while directing mouth breathing through the device and out the front slit opening. With the jaw consciously flexed, resting mouth airflow is comfortably allowed, however, a relaxed sleeper's jaw or a forced inhalation will collapse the slit opening, reducing or stopping airflow, causing the user to breath through their nose, thereby preventing the necessary mouth airflow needed to support snoring.

### BRIEF DESCRIPTION OF THE DRAWINGS

The present invention may be understood from the description of the preferred embodiment which follows and from the accompanying drawings. The drawings are hereby expressly made a part of the specification.

FIG. 1 is a rear perspective view of the anti-snoring device;

FIG. 2 is a rear elevation view of the anti-snoring device depicted in FIG. 1, in the partially expanded conformation it would assume when in use and supporting a sleeping users jaw;

FIG. 3 is a rear elevation, exploded view of the anti-snoring device depicted in FIG. 1;

FIG. 4 is a top planar view of a pre-assembly pattern of the anti-snoring device depicted in FIG. 1.

FIG. 5 is a rear perspective view of an alternate embodiment of the anti-snoring device;

FIG. 6 is a top planar view of a pre-assembly pattern of the anti-snoring device depicted in FIG. 5;

FIG. 7 is a top planar view of the anti-snoring device with a partial peel back view of the adhesive layer;

FIG. 8 is a rear elevation, exploded view of the anti-snoring device depicted in FIG. 7;

FIG. 9a is a right side view of the anti-snoring device depicted in FIG. 7, as it would appear prior to use, with the protective peel-able backing still attached;

FIG. 9b is a right side view of the anti-snoring device depicted in FIG. 5, in the partially expanded conformation it would assume when in use and supporting a sleeping users jaw;

FIG. 10 is a right side view of the anti-snoring device depicted in FIG. 7, inserted between a user's lips for sleep, but prior to any expansion due to a dropping jaw, and featuring an aromatic strip on the top front of the device;

FIG. 11 is a rear elevation view of the anti-snoring device depicted in FIG. 5, in the partially expanded conformation it would assume when in use and supporting a sleeping users jaw;

FIG. 12 is a cross-sectional view of the anti-snoring device depicted in FIG. 1, on the line 12-12 of FIG. 11, depicting the restriction of mouth airflow by the device when in the partially expanded conformation it would assume when in use and supporting a relaxed or sleeping users jaw;

FIG. 13 is a cross-sectional view of the anti-snoring device depicted in FIG. 1, on the line 12-12 of FIG. 1, depicting the device response of increased mouth airflow when in the further expanded conformation it would assume when in use and with a consciously flexed jaw;

FIG. 14 is a cross-section view of the anti-snoring device depicted in FIG. 1, on the line 12-12 of FIG. 11, depicting the device response of collapse and decreased airflow to a forced inhalation when in use and supporting a relaxed or sleeping users jaw;

### DETAILED DESCRIPTION OF THE INVENTION

Reference is made herein to the attached drawings. Like reference numerals are used throughout the various drawings to depict like or similar elements of the claimed anti-snoring device. For the purpose of presenting a brief and clear discussion of the anti-snoring device, the preferred embodiment and one alternate configuration will be discussed primarily as depicted. This is for representative purposes only and should not be construed as limiting in any manner.

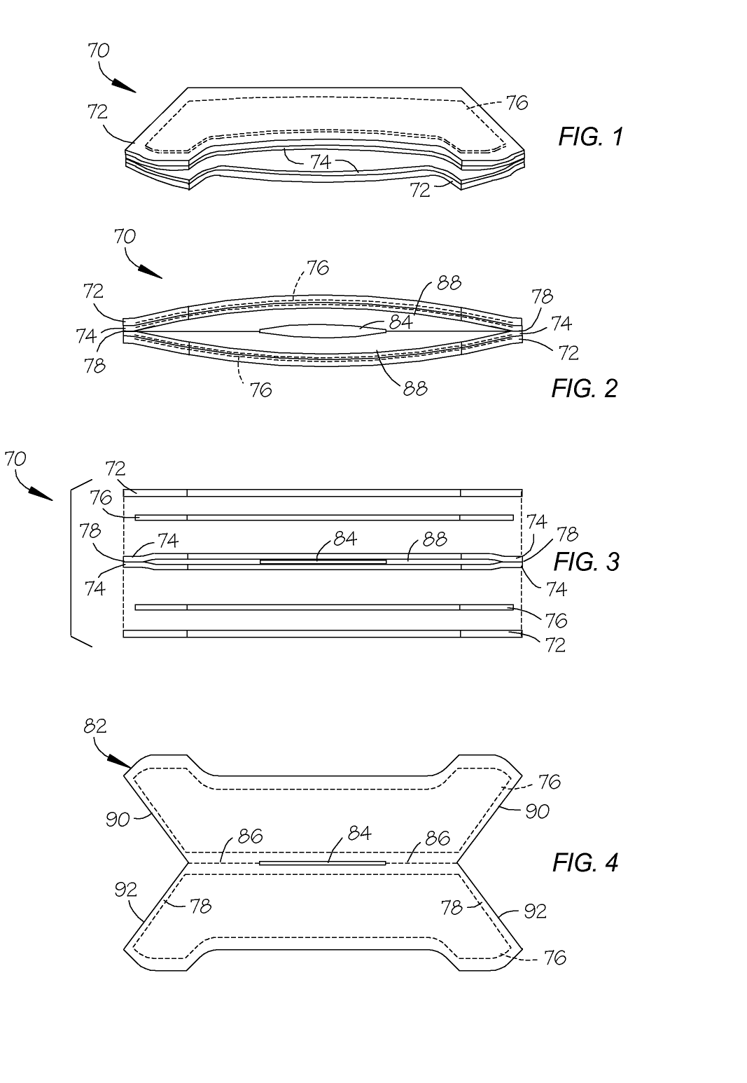

Referring now to FIG. 1, the preferred embodiment 70 of the anti-snoring device is seen as it would appear when held by the user prior to placement between the lips for use, however in a partially expanded conformation. Exterior surface layer 72 and the interior surface layer 74 of the pocket shaped construction 70 are comprised of a flexible material, with two resilient panels 76 being sandwiched in between them, one in the upper portion of the pocket shaped construction 70 seen by hidden view, and one in the lower portion not seen in this view. The inner and outer layers are bonded directly to the resilient panels 76 and to each other at the perimeter of the panels 76, creating an integral tri-layer framework of which the pocket shaped construction 70 is made. The outward facing surface of exterior surface layer 72 is comprised of a pressure sensitive adhesive cushion which substantially covers the lip contact area of the device 70, providing a releasable attachment to the users lips.

FIG. 2 is a rear elevation view of the anti-snoring device 70 in the partially expanded conformation it would assume when in use and supporting a sleeping user's jaw. As can be seen by hidden view, resilient panels 76 are sandwiched between exterior surface layer 72 and interior surface layer 74, one between the upper surfaces and one between the lower surfaces, providing a resilient structure to the pocket. The weight of the sleeping user's jaw causes the resilient panels 76 to bow out away from each other, causing the rear pocket opening 88 and the front slit opening 84 to both expand. The resilient panels are lively enough to hold a sleeping user's jaw from opening wide enough to allow the required mouth airflow for snoring. Pocket edge seams 78 are bonded together, thereby binding the outside ends of resilient panels 76 together, and causing them to frame the rear pocket opening 88.

FIG. 3 provides a rear elevation, exploded view of the anti-snoring device for a more detailed view of the various layers of the device 70 previously discussed. The front slit opening 84 can be seen through the rear pocket opening 88 of the device 70. Pocket edge seams 78 are also clearly visible where they attach the upper and lower interior surfaces 74 together, thereby securing the upper half and lower half of the pocket together. The resilient panels 76 and the exterior surface layers 72 are also clearly seen. This preferred embodiment 70 is shown ready to use, with the overlying peel-off protective layer 138 already removed, which can be seen in FIGS. 7 and 8.

Referring now to FIG. 4, the preferred embodiment 70 of the anti-snoring device is seen as it would appear as a pre-assembly pattern 82. The pattern 82 is cut from a lightweight flexible material which is preferably breathable and waterproof and has a slit opening 84 cut and centered along its long midline 86. The midline 86 divides the pattern 82 into a top portion 90 and a bottom portion 92. Bottom portion 92 has adhesive placed along its outside edges 78 so that when top portion 90 is folded down on midline 86 and upon slit opening 84, the configuration seen in FIG. 1 is formed, creating a pocket shaped construction which resembles a canoe lying on its side, with the unconnected rear edges of the pocket forming an opening 88, as can be seen in FIG. 2, which faces into the user's mouth opposite the slit opening 84, also seen in FIG. 2, in the front edge of the device 70.

The preferred embodiment of the device 70 is sizeably adapted and shaped to fit between the lips of the user while lying lengthwise atop the user's lower lip, and is approximately five-eights inch to three-quarters inches wide and one and one-half inch to two and one-quarter inches long with the slit opening 84 in the front edge of the device 70, being about five-eighths inch to three-quarters inches long. The pressure sensitive adhesive cushion layer 72 allows the top and bottom surfaces of the device 70 to be release-ably attached to the user's upper and lower lips, comfortably sealing the lips about the rear pocket opening 88 of the device 70, and directing substantially all mouth breathing through the device 70 and the front slit opening 84.

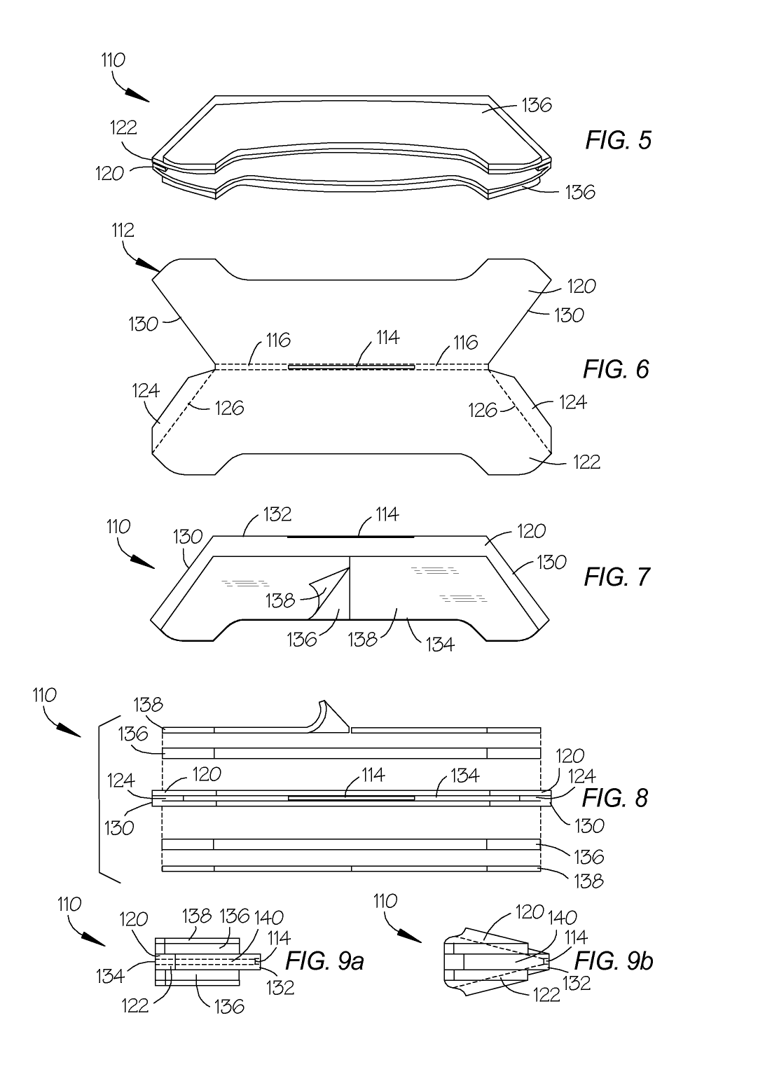

Referring now to FIG. 5, an alternate configuration 110 of the anti-snoring device is seen as it would appear when held by the user prior to placement between the lips for use, however in a partially expanded conformation.

Referring now to FIG. 6, the alternate configuration 110 of the anti-snoring device is seen as it would appear as a pre-assembly pattern 112. The pattern of this alternate embodiment 112 is cut from a resilient material, such as waterproof paper or another suitable semi-rigid, semi-resilient material and has a slit opening 114 cut and centered along its long midline 116. The midline 116 divides the pattern 112 into a top panel 120 and a bottom panel 122. Bottom panel 122 has a fold tab 124 on each end which folds over on fold tab line 126. The pattern may make use of fold tabs on either the top or the bottom panel, or may alternate fold tabs with one on the top panel and one on the bottom panel. Once fold tabs 124 are folded over, then top panel 120 is folded down on midline 116 and upon slit opening 114, to form the configuration seen in FIG. 7. Fold tabs 124 are attached to the opposing panel folded down on top of them, in this case top panel 120, by glue, tape or other suitable means, to join the two panels along their side edges 130.

Referring now to FIG. 7, depicting the alternate configuration 110 of the device, panels 120, 122, being joined along their front edge 132 by the midline 116 fold, and also joined along their side edges 130, form a pocket shaped construction, as can be seen in FIG. 5, with the unconnected rear edges 134 of the pocket forming an opening which faces into the user's mouth, opposite the slit opening 114 in the front edge 132 of the device 110. A pressure sensitive adhesive cushion 136 layer substantially covers the lip contact area of the top panel's 120 and bottom panel's 122 outward face, as can also be seen in FIG. 5. A peel-off protective paper 138 layer covers over the adhesive cushion 136 layer which can be seen partially peeled back.

FIG. 8 provides a rear elevation, exploded view of the alternate configuration 110 of anti-snoring device for a more detailed view of the various layers of the device 110 previously discussed. The slit opening 114 can be seen through the rear pocket opening 134 of the device 110. Fold tabs 124 are also clearly visible where they attach to top panel 120 along the side edges 130. The adhesive cushion layer 136 and the overlying peel-off protective paper 138 layer are also clearly seen.

FIG. 9a. provides a right side view of the alternate configuration 110 of device, as it would appear prior to use, with the protective peel-able paper 138 still attached. The air channel 140 extending from the rear pocket opening 134 to the slit opening 114 in the front edge 132 is clearly seen in an un-expanded state. In this configuration almost no airflow is allowed through the device. When in use and supporting a user's jaw, the top panel 120 and bottom panel 122 bow out from each other causing air channel 140 to also expanded, as can be seen in FIG. 9b. and also in FIG. 5. In this configuration, where the mouth and jaw are relaxed, the device allows from 10-30% of resting mouth airflow through the device, with the balance of needed airflow coming from nasal breathing, which is enough to give a sleeping user a comfort level whereby they are not even aware that they have the device on.

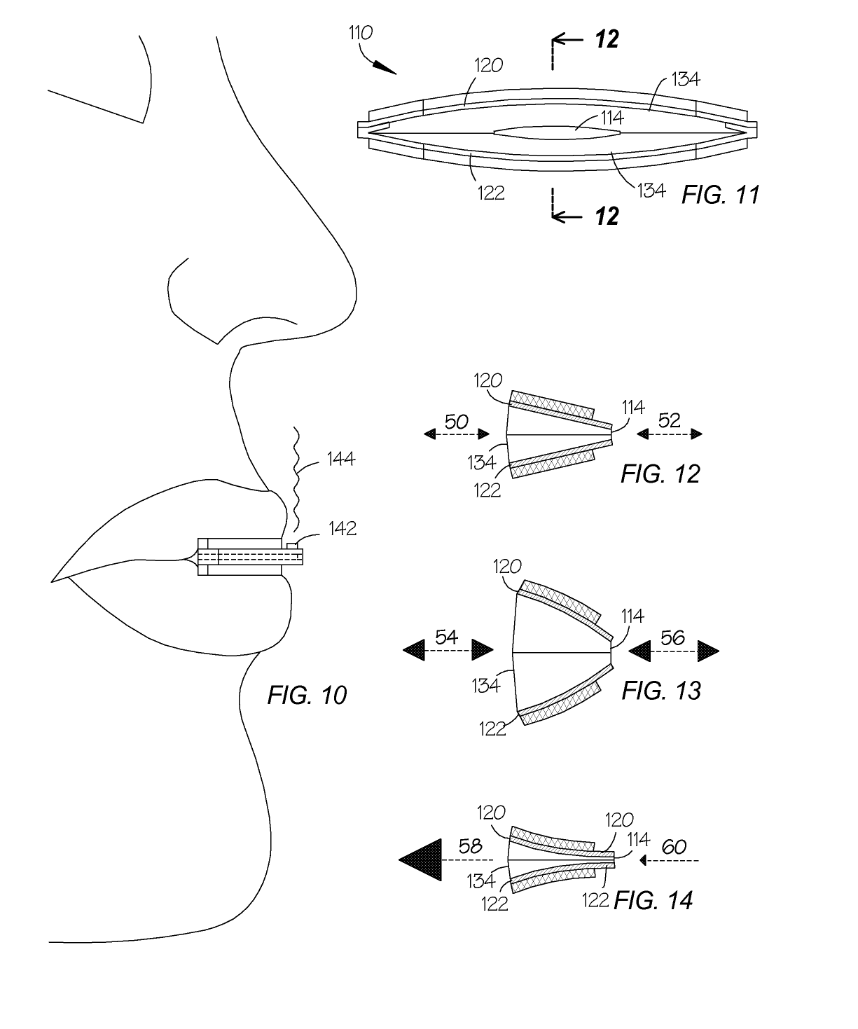

Referring now to FIG. 10, the alternate configuration 110 of the device, is seen inserted between a user's lips for sleep, but prior to any expansion due to a dropping jaw, and featuring an aromatic strip 142 on the front end of top panel 120 of the device. The aromatic strip 142 is conveniently located in close proximity to and in alignment with the user's nostrils, for maximum effectiveness of the aromatic vapors 144.

FIG. 11 is a rear elevation view of the alternate configuration 110 of the device, in the partially expanded conformation it would assume when in use and supporting a sleeping user's jaw. As can be seen, top panel 120 and bottom panel 122 bow out away from each other, causing the rear pocket opening 134 and the front slit opening 114 to both expand. The regulating effects of the Anti-Snoring device, including the preferred embodiment 70, as seen in FIGS. 1-4, and the alternate configuration 110, FIGS. 5-11, on airflow can be better understood by cross-sectional views of the device 110 starting from this configuration, as can be seen in FIGS. 12, 13 and 14.

Referring now to FIG. 12, the device 110 is seen in cross-section, being partially expanded, where a sleeping user's dropping jaw has pulled the pocket opening 134 apart by the bowing out of the top and bottom panels 120, 122, allowing for 10-30% of resting mouth airflow, where airflow volume at the rear pocket opening 50 will be equal to the airflow volume at the front slit opening 52.

Referring now to FIG. 13, the device 110 is seen in cross-section when mild flexing of the user's jaw has pulled the pocket opening 134 wider apart by the further bowing out of the top and bottom panels 120, 122, allowing for increased airflow when consciously desired by the user, where airflow volume at the rear pocket opening 54 will be equal to the airflow volume at the front slit opening 56 up to 80-100% of resting mouth airflow and alleviating any feelings of claustrophobia the restricted airflow may have caused.

Referring now to FIG. 14, the device 110 is seen in cross-section when a forced inhalation by the user has created negative pressure within the device 110 and pulled the pocket opening 134 partially closed by the bowing in of the top and bottom panels 120, 122, and causing the front slit opening 114 to collapse causing a decrease in or shutoff of allowed airflow, where airflow volume demand at the rear pocket opening 58 will greatly exceed the airflow volume allowed at the front slit opening 60, thereby causing the user to shift to nasal breathing and preventing mouth inhalations from reaching the critical airflow volumes required to support snoring.

The method and embodiment of the present invention shown and discussed are by way of illustration and not of limitation, and a wide variety of equivalent methods and embodiments may be made without departing from the spirit or scope of the invention.

What is claimed is:

1. An anti-snoring device, comprising:
an inter-lip oral breath volume regulating means, whereby a user's resting mouth airflow is comfortably allowed when the user's jaw is consciously flexed, but whereby, a relaxed sleeper's jaw or a forced inhalation will cause a significant decrease or stoppage of mouth airflow, thereby causing the user to shift to nasal breathing and preventing mouth inhalations from reaching the critical airflow volumes required to support snoring; and
a lip attachment means, whereby said inter-lip breath volume regulating means is secured between the lips and attached thereto, without intruding beyond the lips and into the mouth or protruding excessively therefrom, and holding the lips and mouth closed about said inter-lip oral breath regulating means, preventing the lower jaw from dropping open during sleep, and whereby mouth breathing is directed through said inter-lip breath volume regulating means.

2. The anti-snoring device according to claim 1, further comprising an aromatic substance delivery means attached to the portion of said inter-lip oral breath regulating means protruding beyond the attached lips, whereby an aromatic substance is provided for nasal inhalation by the user for a soothing scent or to provide dilation of the nasal cavity and sinus tissues to further promote comfortable nasal breathing while sleeping.

3. The anti-snoring device according to claim 1, wherein said inter-lip breath volume regulating means is comprised of a pair of elongated resilient panels, each having a top and a bottom surface and front, rear and side edges, one said panel lying atop the other in the horizontal plane, and sizeably adapted and shaped to fit between the lips of the user while lying lengthwise atop the user's lower lip, said panels being attached to each other along the majority of said elongated front edges in a hinge like manner, but leaving an unconnected portion near the center of said front edge thereby creating a collapsible slit opening, said panels being joined to each other along said side edges, thereby forming a pocket shaped construction with said front edge facing outwards from the lips, and said unconnected rear edges of said pocket forming an opening into the user's mouth opposite said slit opening in said front edge, whereby mild flexing of the mouth when attached thereto will pull said pocket opening apart by the bowing out of said resilient panels, allowing for comfortable airflow during resting mouth breathing, but whereby a relaxed sleeper's jaw will allow said resilient panels to partially pull back together, causing said slit opening in said front edge of said device to partially collapse, significantly reducing mouth airflow, and whereby a forced inhalation and the concomitant negative pressure created thereby will cause said resilient panels to bow in, causing said slit opening in said front edge of said device to collapse, causing a shutoff of allowed mouth airflow.

4. The anti-snoring device according to claim 3, wherein said lip attachment means is comprised of an adhesive layer substantially covering the lip contact area of each said panel's outward face, whereby said panels are release-ably attached to the user's upper and lower lips, sealing the lips about said rear pocket opening of said device, and directing substantially all mouth breathing through said device and said slit opening in said front edge of said device.

5. The anti-snoring device according to claim 4, wherein said adhesive layer is further comprised of a pressure sensitive adhesive cushion to provide a more comfortable fit for the user.

6. The anti-snoring device according to claim 5, further comprising a peel-off protective layer over said adhesive layer.

7. An anti-snoring device, comprising:
a pair of elongated resilient panels, each having a top and a bottom surface and front, rear and side edges, one said panel lying atop the other in the horizontal plane, and sizeably adapted and shaped to fit between the lips of the user while lying lengthwise atop the user's lower lip, said panels being attached to each other along the majority of said elongated front edges in a hinge like manner, but leaving an unconnected portion near the center of said front edge thereby creating a collapsible slit opening, said panels also being joined to each other along said side edges, thereby forming a pocket shaped construction with said front edge facing outwards from the lips, and said unconnected rear edges of said pocket forming an opening into the users mouth opposite said slit opening in said front edge, and allowing for the regulation of the user's breath volume, whereby mild flexing of the mouth will pull said pocket opening apart by the bowing out of said resilient panels, allowing for comfortable airflow during resting mouth breathing, but whereby a relaxed sleeper's jaw will allow said resilient panels to partially pull back together, causing said slit opening in said front edge of said device to partially collapse, significantly reducing mouth airflow, and whereby a forced inhalation and the concomitant negative pressure created thereby will cause said resilient panels to bow in, causing said slit opening in said front edge of said device to collapse, causing a shutoff of allowed mouth airflow, thereby causing the user to shift to nasal breathing and preventing mouth inhalations from reaching the critical airflow volumes required to support snoring; and
a pressure sensitive adhesive cushion layer substantially covering the lip contact area of each said panel's outward face, whereby said panels are release-ably attached to the user's upper and lower lips, comfortably sealing the lips about said rear pocket opening of said device, and directing substantially all mouth breathing through said device and said slit opening in said front edge of said device; and
a peel-off protective layer over said adhesive layer.

8. The anti-snoring device according to claim 7, further comprising an aromatic substance delivery strip attached to a portion of the front end of said device protruding beyond said lip attachment adhesive layer, whereby an aromatic substance is provided for nasal inhalation by the user for a soothing scent or to provide dilation of the nasal cavity and sinus tissues to further promote comfortable nasal breathing while sleeping.

9. The anti-snoring device according to claim 7 in which said slit opening in said front edge is about five-eighths inch to three-quarters inches long.

10. The anti-snoring device according to claim 7 in which said device is approximately five-eights inch to three-quarters inches wide and one and one-half inch to two and one-quarter inches long.

11. The anti-snoring device according to claim 7 in which said resilient panels are comprised of material selected from: plastic, paper, coated paper, waterproof paper, lightweight cardstock, lightweight composites, resinous material or a combination thereof.

12. The anti-snoring device according to claim 7, in which said pocket shaped construction is formed by a layer of continuous flexible material, said pocket having an upper and a lower inner surface and an upper and lower outer surface, said resilient panels being bonded to said inner surfaces of said flexible pocket material, one panel bonded to said upper inner surface and one panel bonded to said lower inner surface, such that one said panel lies atop the other said panel in the horizontal plane, forming the new inner surfaces of said pocket, and in which said outer upper and outer lower surfaces of said flexible pocket material forms said adhesive cushion layer.

13. The anti-snoring device according to claim 12, further comprising an additional layer of said continuous flexible material, forming an inner pocket within said pocket and being bonded over and to said resilient panels and said inner surfaces of said pocket construction, thereby forming an integral tri-layer pocket construction with said resilient panels being encased within said upper and lower pocket surfaces.

14. The anti-snoring device according to claim 13, further comprising an aromatic substance delivery strip attached to a portion of the front end of said device protruding beyond said lip attachment adhesive layer, whereby an aromatic substance is provided for nasal inhalation by the user for a soothing scent or to provide dilation of the nasal cavity and sinus tissues to further promote comfortable nasal breathing while sleeping.

15. The anti-snoring device according to claim 13 in which said slit opening in said front edge is about five-eighths inch to three-quarters inches long.

16. The anti-snoring device according to claim 13 in which said device is approximately five-eights inch to three-quarters inches wide and one and one-half inch to two and one-quarter inches long.

17. The anti-snoring device according to claim 13 in which said resilient panels are comprised of material selected from: plastic, paper, coated paper, waterproof paper, lightweight cardstock, lightweight composites, resinous material or a combination thereof.

18. The anti-snoring device according to claim 13 in which said continuous flexible material is comprised of material selected from: a woven fabric material, a non-woven fabric material, a woven thermoplastic fabric, a non-woven thermoplastic fabric, or a combination thereof.

19. The anti-snoring device according to claim 13, in which said protective layer has a cut across its surface to aid in removing it from said adhesive layer.

20. The anti-snoring device according to claim 13 in which the adhesive layer is hypoallergenic.
